(19) United States
(12) Patent Application Publication
(10) Pub. No.: US 2006/0025481 A1
(43) Pub. Date: Feb. 2, 2006

(54) Process for preparation of probucol derivatives and polymorphic forms thereof

(76) Inventor: Matthew L. Strange, (US)

Correspondence Address: KING & SPALDING LLP, 191 PEACHTREE STREET, N.E., 45TH FLOOR, ATLANTA, GA, 30303-1763, US

(21) Appl. No.: 11/054,694

(22) Filed: Feb. 9, 2005

Related U.S. Application Data
(60) Provisional application No. 60/560,730, filed on Feb. 9, 2004.

(51) Int. Cl. C07C 323/31; A61K 31/19

(52) U.S. Cl. 514/568; 562/9

(57) ABSTRACT

A process is described for the preparation of polymorphic forms of water-soluble derivatives of probucol compounds. The process generally includes reacting a probucol compound with a solution of alkali metal oxide, reacting the salt mixture with a solution of an alkyl or aryl halo-substituted carboxylate, hydrolyzing the resulting compound; and separating a crystalline, polymorphic form of the compound from the hydrolyzing mixture. Polymorphic forms of the probucol compounds are also described.

### REFERENCE TO PRIOR APPLICATION

This application claims priority to U.S. Provisional Application No. 60/560,730, filed Feb. 9, 2004, entitled “Process For Preparation Of Probucol Derivatives And Polymorphic Forms Thereof” and is a continuation-in-part of U.S. patent application Ser. No. 10/730,178, filed Dec. 8, 2003, entitled “Process for the Preparation of Probucol Derivatives and Polymorphic Forms Thereof.”

### FIELD OF INVENTION

The invention describes certain derivatives of 4,4′-(isopropylidenedithio) bis[2,6-di-tert-butylphenol], known by the generic name “probucol,” and processes for the preparation of probucol derivatives and polymorphic forms thereof.

### BACKGROUND OF THE INVENTION

Probucol is a well-known antioxidant that is related to antioxidant compounds such as tertiary butyl-4-hydroxyanisole, 2,6-di-tertiary butyl-4-methylphenol and the like. These compounds are used in food and food products to prevent oxidative deterioration.

The compound probucol is represented by the following structural Formula:

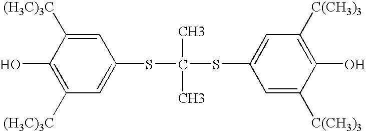

The preparation of this compound is a multistep process, starting by reacting a solution of the appropriately-substituted 4-mercaptophenol with acetone, in the presence of a catalytic amount of hydrochloric acid. Probucol precipitates from the reaction mixture and is readily separated and purified. The reaction is described in detail in U.S. Pat. No. 3,862,332 (Barhhart et al). Similarly, probucol and certain of its derivatives are also described in U.S. Pat. No. 3,485,843 (Wang), U.S. Pat. No. 3,576,833 (Neuworth) and U.S. Pat. No. 4,985,465 (Handler). However, because probucol is minimally soluble in water, its use is limited.

In order to avoid the low water solubility problems associated with use of probucol, water-soluble derivatives have been prepared. For example, U.S. Pat. No. 5,262,439 (Parthasarathy) discloses several classes of probucol derivatives that are described as being water-soluble. Some of the compounds disclosed in this reference have polar or charged functionalities attached to an ester group, e.g., a carboxylic acid, amide, amino, or aldehyde. The method disclosed for preparing these water-soluble probucol compounds involves the reaction of probucol with the carboxylic acid anhydride compound bearing the desired polar or charged functionality in the presence of a catalyst.

Similarly, U.S. Pat. Nos. 6,323,359 and 6,548,699 also disclose water soluble derivatives of probucol. The compounds set forth in the former patent are produced by a process involving the reaction of a probucol dianion with carboxylic acid anhydrides. The compounds disclosed in U.S. Pat. No. 6,548,699 are synthesized by reaction of probucol with, inter alia, halo-substituted aliphatic esters.

The prior art processes for preparing alkylated probucol derivatives do not produce appreciable yields of probucol derivatives.

It is therefore an object of the invention to provide a novel process for efficiently preparing probucol derivatives in high yields.

### SUMMARY OF THE INVENTION

The invention provides a new process for preparing probucol derivatives in high yields as well as new polymorphic forms of probucol derivatives. The process provides includes a first reaction of probucol with an alkali metal or ammonium-containing compound to produce, as a mixture, the mono- and dialkali metal salts of probucol, e.g, the mono- or dialkali metal salt of 4,4′-(isopropylidenedithio) bis[2,6-di-tert-butylphenol]. In a second step, this anionic intermediate mixture is reacted with an alkyl or aryl halo-substituted alkyl or alkenyl carboxylate, displacing the alkali metal cation(s) and substituting a proton, an alkyl or alkenyl carboxylate alkyl or aryl ester group for each of such cations. The compounds that are produced from such reaction are a probucol monoalkyl or monoalkenyl carboxylic acid ester and probucol dialkyl or dialkenyl carboxylic acid ester. Ester hydrolysis results in two products, the salts of probucol monoalkyl or monoalkenyl carboxylic acid and of the probucol dialkyl or the dialkenyl carboxylic acid. These water-soluble probucol compounds are then separated from the resulting reaction mixture. Protonation of the separated compounds produces a free carboxylic acid derivative. More than one carboxylic acid group may be present in the final probucol product if the compound used in the second step of process of this invention is a dibasic acid derivative.

It has also been discovered that the water soluble derivatives of probucol prepared in accordance with the process of the present invention produce one or more crystalline, polymorphic compounds. Such crystalline polymorphic forms of these probucol derivatives are stable.

The present invention also includes pharmaceutical compositions comprising an effective amount of one or more of the crystalline polymorphic forms of the soluble derivatives of probucol along with pharmaceutically acceptable carriers. The invention also includes the use of such compositions for the treatment of diseases of the vasculature, inflammatory and cardiovascular diseases, and as serum cholesterol-lowering agents for treating patients suffering from hypercholesterolemia, as effective antiviral compounds and as agents to treat arrhythmia.

### BRIEF DESCRIPTION OF THE DRAWINGS

FIG. 1 is a powder x-ray diffraction pattern of the novel polymorphic Form A of the probucol compound 2-[4-[[1-[3,5-bis(1,1-dimethylethyl)-4-hydroxyphenyl]thio]-1-[methylethyl]thio]-2,6-bis(1,1-dimethylethyl) phenoxy] ethanoic acid

FIG. 2 is a powder x-ray diffraction pattern of the novel polymorphic Form B of the probucol compound 2-[4-[[1-[3,5-bis(1,1-dimethylethyl)-4-hydroxyphenyl]thio]-1-[methylethyl]thio]-2,6-bis(1,1-dimethylethyl) phenoxy] ethanoic acid.

FIG. 3 is a powder x-ray diffraction pattern of the novel polymorphic Form C of the probucol compound 2-[4-[[1-[3,5-bis(1,1-dimethylethyl)-4-hydroxyphenyl]thio]-1-[methylethyl]thio]-2,6-bis(1,1-dimethylethyl) phenoxy] ethanoic acid.

FIG. 4 is a powder x-ray diffraction pattern of the novel polymorphic Form D of the probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid

FIG. 5 is a powder x-ray diffraction pattern of the novel polymorphic Form E of the probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid

FIG. 6 is a powder x-ray diffraction pattern of the novel polymorphic Form F of the probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid

FIG. 7 is a powder x-ray diffraction pattern of the novel polymorphic Form G of the probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid.

FIG. 8 is a powder x-ray diffraction pattern of the novel polymorphic Form H of the probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid.

FIG. 9 is a powder x-ray diffraction pattern of the novel polymorphic Form I of the probucol compound probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid.

FIG. 10 is a powder x-ray diffraction pattern of the novel polymorphic Form J of the probucol compound probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid

FIG. 11 is a powder x-ray diffraction pattern of the novel polymorphic Form K of the probucol compound probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid.

FIG. 12 is a powder x-ray diffraction pattern of the novel polymorphic Pattern L of the probucol compound probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid

FIG. 13 is a powder x-ray diffraction pattern of the novel polymorphic Form M of the probucol compound probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] ethanoic acid

FIG. 14 is a powder x-ray diffraction pattern of the novel polymorphic Form M of the probucol compound probucol compound 2-[4-[[1-[3,5-bis(1,1-dimethylethyl)-4-hydroxyphenyl]thio]-1-[methylethyl]thio]-2,6-bis(1,1-dimethylethyl)phenoxy] butanoic acid

### DESCRIPTION OF THE PREFERRED EMBODIMENTS

The present invention provides a process for preparing certain water soluble probucol derivatives and to crystalline polymorphic forms of such water soluble probucol derivatives.

As used herein, the term “alkyl” is intended to mean and include the groups that are C₁ to C₈ linear or branched alkyl which include the moieties methyl, ethyl, n-propyl, isopropyl, butyl, sec-butyl, tert-butyl, n-pentyl, 2-methylpentyl, and the like. Any time a range is described in this specification it is intended to independently cover every individual point in the range. Thus, when the range C₁ to C₈ alkyl is described, it is intended to cover any of C₁, C₂, C₃, C₄, C₅, C₆, C₇ or C₈ alkyl.

The term “aryl” is intended to mean and include the aromatic radicals that may be substituted or unsubstituted one or more times by alkyl, nitro or halo which includes phenyl, naphthyl, phenanthryl, anthracenyl, thienyl, pyrazolyl and the like.

The term “alkenyl” is intended to mean and include the groups that are C₃ to C₈ linear or branched alkenyl which include the moieties 1-propenyl, 1-butenyl, 2-butenyl, 1-pentenyl, 2-pentenyl and the like.

The term “alkali metal” is intended to mean those metals in Group I and Ia of the Periodic Table of the Elements such as lithium, potassium, sodium and the like.

The water soluble derivatives of the probucol compounds herein are obtained by reaction of a solution of one or both of the hydroxyl groups of probucol with a compound that forms an alkali metal or ammonium salt of probucol, i.e., the alkali metal or ammonium substitutes for hydrogen at one or both of probucol's hydroxyl groups. The compounds that form these salts are strongly basic reactants. They are illustrated by the alkali metal hydrides, alkali metal hydroxides, alkali metal alkoxides, alkyl ammonium alkoxides or alkyl ammonium hydroxides. Mixtures of these compounds are also useful in producing the desired probucol salts. Potassium is the most preferred alkali metal of these strongly basic reactants used in this step. The solvent used to form the solution of probucol is preferably N-methylpyrrolidone.

This first reaction (step 1) in the process of the present invention produces a mixture of mono- and dianions of the following Formula (where A is proton or an alkali metal cation) 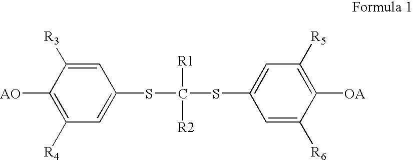
where R₁ and R₂ are the same or different and are independently alkyl, alkenyl or aryl having from 1 to 8 carbon atoms and R₃, R₄, R₅ and R₆ are the same or different and are independently alkyl having from 1 to 4 carbon atoms.

In one embodiment, R₁ and R₂ are the same and are alkyl having from 1 to 8 carbon atoms, most preferably methyl.

In another embodiment, R₃, R₄ R₅ and R₆ are the same and are alkyl having from 1 to 4 carbon atoms. In a specific embodiment, R₃, R₄ R₅ and R₆ are tert-butyl.

In a second step in the process, the mixture of mono- and dianions produced in step 1 is reacted with a solution of an alkyl or aryl halo-substituted alkyl or alkyl halo-substituted alkenyl carboxylate having the Formula:
Halo-CₙH₂ₙ—C(O)OV or Halo-CₙH_{2n′-2}—C(O)OV
where V is alkyl or aryl; Halo is chloro, bromo or iodo; alkyl is C₁ to C₆ linear or branched alkyl; the group CₙH₂ₙ— is a C₁ to C₆ linear or branched saturated hydrocarbon group; the group —CₙH_{2n′-2} is a C₃ to C₆ linear or branched unsaturated hydrocarbon group; n is an integer from 1 to 6; and n′ is an integer from 3 to 6. The reaction yields at least one compound of the Formula: 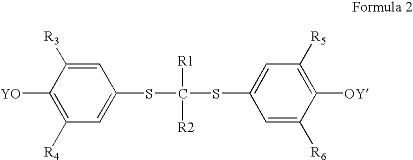
where R₁, R₂, R₃, R₄, R₅ and R₆ are defined above and Y and Y′ are the same or different and are independently hydrogen or the moiety —CₙH₂ₙ—C(O)OV or the moiety —CₙH_{2n′-2}—C(O)OV where V, —CₙH₂ₙ, CₙH_{2n′-2}, n and n′ are as previously defined, with the proviso that Y and Y′ can not both be hydrogen.

In one embodiment, the alkyl halo-substituted alkyl carboxylate compounds used in the reaction of step 2 are the methyl and ethyl esters of halo acetic acid, halo propionic acid and the like where “halo” is chloro, bromo or iodo. In a particular embodiment, the compounds used in the reaction step 2 are bromo substituted, and can particularly be bromo propionic or acetic acid esters.

In one amebodiment, the alkyl halo-substituted alkenyl carboxylate compounds in the reaction of step 2 are the methyl and ethyl esters of halo butene-3-carboxylic acid, halo pentene-4-carboxylic acid and the like where “halo” is chloro, bromo or iodo. In one embodiment, the compounds used in the reaction step 2 are bromo substituted and can particularly be bromo butene-3-carboxylic acid or bromo pentene-4-carboxylic acid esters.

In one embodiment of the above ester compounds include the group —CₙH₂ₙ—, and in particularly embodiments, include the group —CH₂— or —C₃H₆—.

Step 3 of the reaction sequence is the hydrolysis of the ester group to produce the probucol carboxylate compound, i.e., the compound of the Formula: 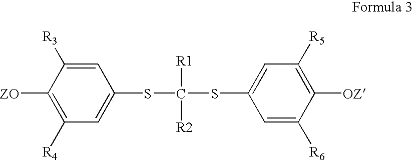
where Z and Z′ are the same or different and are independently hydrogen, or the moiety —CₙH₂ₙ—C(O)O⁻A⁺ or the moiety —C_{n′}H_{2n′-2}—C(O)O⁻A⁺ where A, —CₙH₂ₙ—, —C_{n′}H_{2n,′2-1}, n and n′ are as previously defined, with the proviso that Z and Z′ can not both be hydrogen.

Ester hydrolysis reactions are well known and can be either acid or base catalyzed. In one embodiment, the ester hydrolysis is a base-catalyzed hydrolytic reaction. Such hydrolytic reaction can be carried out in a solvent. Bases used in this hydrolysis step are exemplified by those compounds noted as useful to produce the mixture formed in step 1 and include a compound selected from the group consisting of alkali metal hydroxide, alkali metal alkoxide, alkyl ammonium alkoxide, alkyl ammonium hydroxide and mixtures thereof.

The base catalyzed hydrolysis typically results in the (di)alkali metal or (di)ammonium salt of the probucol derivative (i.e., the hydrogen atom of the carboxylic acid group, Z and/or Z′ is replaced by an alkali metal or ammonium anion). Therefore, in one embodiment that includes a base-catalyzed hydrolysis, the embodiment can also include an acid treatment. In this embodiment, the reaction is followed by an acid treatment to protonate the carboxylate group and give the free acid. Such free acid is easily separated as a crystalline solid.

In step 1 of the process of the present invention, the reactants are admixed in a suitable organic solvent. The mixture of mono- and dialkali metal or ammonium salt of the probucol derivative can readily form in as little as one hour or up to about six hours after such admixing, typically under room temperature conditions. The diphenolate salt may be removed from the reaction solution as a solid (by precipitation and filtration, etc.) and subsequently used in step 2 of the process.

Alternatively, the reaction solution resulting from step 1 of the reaction can be used “as is” for the second step, i.e., without separating the mixture of mono- and dianions. In one embodiment, the salt produced in the first step is treated with a compound that has a group that will react with at least one of the alkali metal probucol phenolates. In this embodiment, in step 2, the halo group of the halo-substituted alkyl or alkenyl carboxylates will react with at least one of the alkali metal or ammonium phenolates to form an alkali metal or ammonium halide by-product and the desired probucol derivative, i.e., the derivative that is substituted with one alkyl or alkenyl carboxylate group. However, it should be noted that because there are two reactive sites available in the mono- and dianionic mixture, either one or both of these sites can be substituted by the incoming halo ester.

The substitution reaction of step 2 is typically carried out from one to about six hours at in an organic solvent. As noted, two probucol derivatives may be formed. In one embodiment, a mono substitution product is formed, where Z and Z′ are different and are independently hydrogen and the moiety —CₙH₂ₙ—C(O)OV or the moiety —C_{n′}H_{2n′-2}—C(O)OV where V, —CₙH₂ₙ—, —C_{n′}H_{2n′-2}, n and n′ are as previously defined. In a separate embodiment, the disubstitution product is formed, where Z and Z′ are the same and are the moiety —CₙH₂ₙ—C(O)OV or the moiety —C_{n′}H₂ₙ₋₂—C(O)OV where V, —CₙH₂ₙ—, —C_{n′}H_{2n′-2}, n and n′ are as previously defined.

As noted, step 3 can be a base hydrolysis (in solution) and can be carried out with an alkali metal hydroxide. In one embodiment, the alkali metal hydroxide is potassium hydroxide. In another embodiment, aqueous N-methylpyrrolidone is the solvent. By this procedure, the alkali metal or ammonium salts of the above compounds, i.e., compounds where the hydrogen atom of the carboxylic acid group of Z and Z′ are replaced by an alkali metal or ammonium anion, can be produced. In one embodiment, ethyl bromoacetate in step 2 is used for one of the reactants. In another embodiment, potassium hydroxide is used as the basic catalyst. In these embodiments, the compounds produced in this step 3 hydrolysis typically bear one or two groups of the Formula —CH₂—C(O)O⁻K⁺. When the alkali metal anion is potassium, the mono substitution product can be formed, i.e., where Z and Z′ are different and are independently potassium and the moiety —CₙH₂ₙ—C(O)O⁻ K⁺ or the moiety —C_{n′}H_{2n′-2}—C(O)OK⁺ where —CₙH₂ₙ—, —C_{n′}H_{2n′-2}, n and n′ are as previously defined. In another embodiment, the disubstitution product is formed, i.e., where Z and Z′ are the same and are the moiety —CₙH₂ₙ—C(O)O⁻K⁺ or the moiety —CₙH_{2n′-2}—C(O)O⁻K⁺ where —CₙH₂ₙ—, —CₙH_{2n′-2} n and n′ are as previously defined. Treatment of these alkali metal salts with an organic or inorganic acid can produce the free carboxylic acid derivatives.

In one process for hydrolyzing the alkali metal salts of step 3 (and any residual probucol-related impurities from the earlier steps of the reaction), the base-catalyzed hydrolysis is typically carried out in an aqueous N-methylpyrrolidone solution. The inorganic impurities that result from the base-catalyzed hydrolysis are then separated from the desired organic hydrolysis products. In one embodiment, the route to accomplish such separation is by first separating (typically by extraction) with a nonpolar, typically hydrocarbon, solvent which is effective in removing unreacted probucol from the hydrolysis reaction mass. The probucol derivatives preferentially partition into the aqueous polar solvent system. These probucol derivatives are then selectively extracted from the hydrolysis mixture at a suitable pH, with a non-polar organic solvent. The impurities remaining in the original nonpolar organic solution, mostly unreacted probucol, are retained for later reuse. The nonpolar hydrocarbon solvents of use in this separation step are preferably toluene or the C₅ to C₁₀ linear or branched hydrocarbons, most preferably n-pentane, n-hexane or n-heptane.

The separation of the mono and dialkali metal or ammonium salts from the mixture produced in the second separation can be accomplished by selectively extracting the dialkali metal or ammonium salts from the nonpolar organic solvent solution. In this third separation, an aqueous salt solution (such as aqueous potassium bicarbonate) selectively dissolves the dialkali metal or ammonium probucol derivative, thereby producing a solution of the mono alkali metal or ammonium salt of the probucol derivative and an aqueous phase which is the di alkali metal or ammonium salt of the probucol derivative. Extraction, decantation, etc. separates these two phases.

The free carboxylic acid may be readily produced from the solution containing the mono alkali metal or ammonium salt by acidification with a dilute aqueous solution of an inorganic acid, such as hydrochloric acid, phosphoric acid, etc. In one embodiment, the acid for such hydrolysis is dilute phosphoric acid, particularly 2.5% phosphoric acid.

The crystalline structure of the desired product produced by the process of the present invention, i.e., the compounds of the Formula 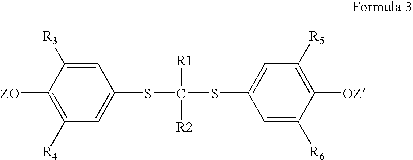
where Z and Z′ are different and are independently hydrogen, the group —CₙH₂ₙ—C(O)OH or the group —CₙH_{2n′-2}—C(O)OH where —CₙH₂ₙ—, —CₙH_{2n′-2}, n and n′ are as previously defined, can be influenced by the reaction conditions and solvent used to precipitate the free carboxylic acid products.

The prior art preparation of the compounds of the present invention produces amorphous products. Unlike processes for preparation of probucol derivatives known in the art, the process of the present invention produces crystalline polymorphic compounds of Formula 3, where R₁, R₂, R₃, R₄, R₅, R₆, Z and Z′ are as previously defined.

In one embodiment of the invention, a solution of the amorphous water soluble derivative of any one of the probucol compounds having the above Formula 3 can be heated for a time and at a temperature sufficient to produce the polymorphic form of such compound. The selection of the solvent and/or the temperature controls which of the numerous polymorphic forms are produced.

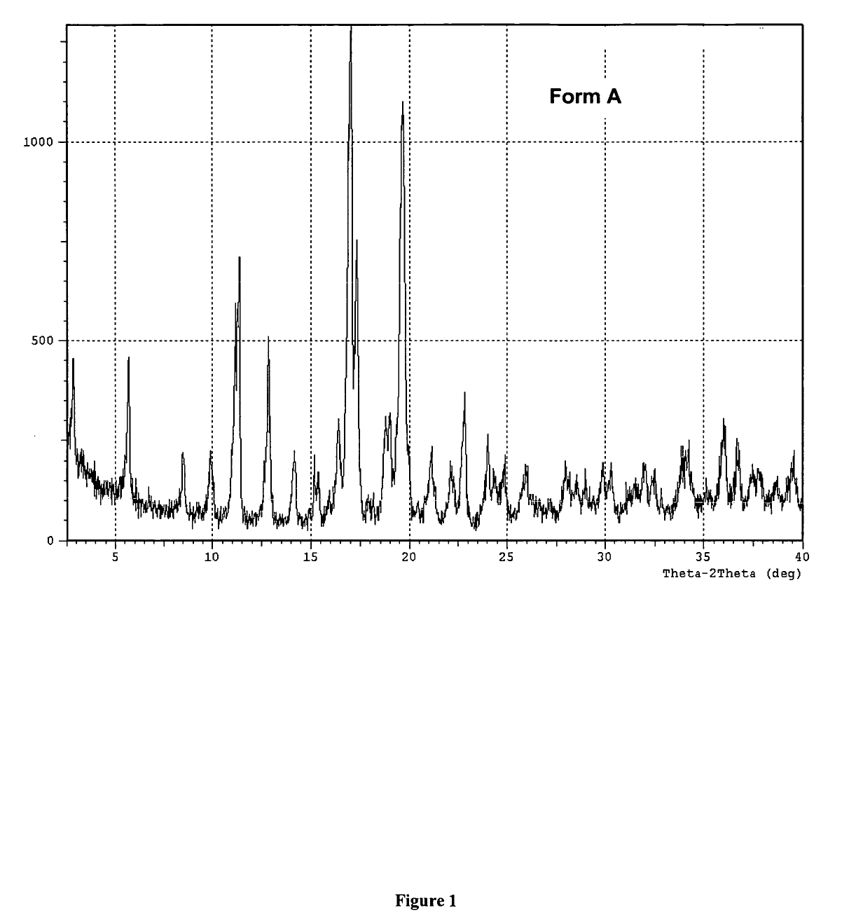

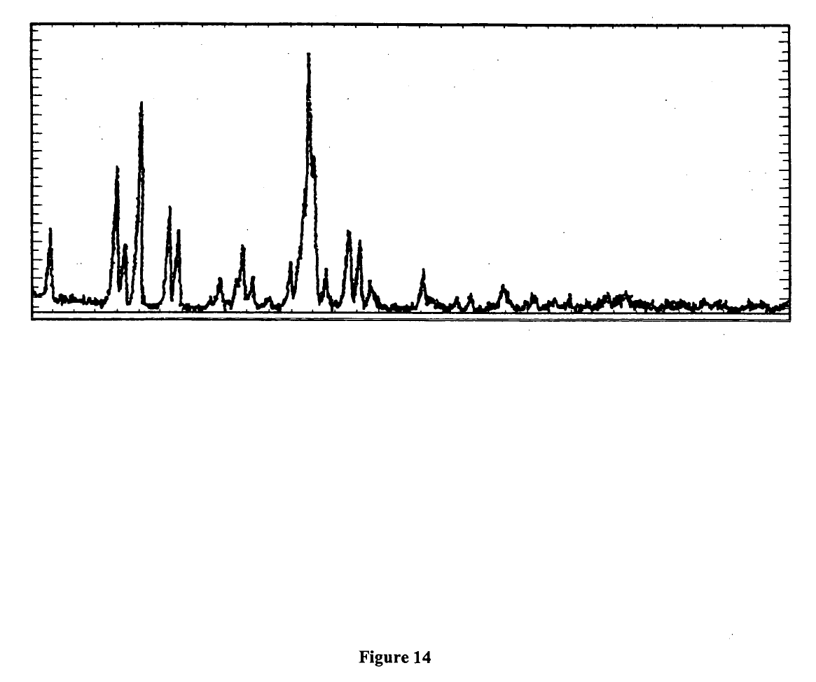

In one embodiment, polymorphic compounds produced by the process of the present invention are those of Formula 3, where R₁, R₂, R₃, R₄, R₅ and R₆ are previously defined and Z and Z′ are different and are hydrogen and the group —CH₂—C(O)OH or the group —(CH₂)₃—C(O)OH. The former compound, 2-[4-[[1-[3,5-bis(1,1-dimethylethyl hydroxyphenyl]thio]-1-[methylethyl]thio]-2,6-bis(1,1-dimethylethyl)phenoxy] ethanoic acid has 13 novel polymorphic forms (polymorphic Forms A, B, C, D, E, F, G, H, I, J, K, L and M), which are described by powder X-ray diffraction patterns shown the FIGS. 1 thru 13 as well as their method of preparation (below). The latter compound i.e., 2-[4-[[1-[3,5-bis(1,1-dimethylethyl hydroxyphenyl]thio]-1-[methylethyl]thio]-2,6-bis(1,1-dimethylethyl)phenoxy] butanoic acid has one defined polymorphic form. It is Form Al and described in FIG. 14.

In the case of FIGS. 1 to 13, the X-ray diffraction (XRPD) analyses were performed using a Shimadzu XRD-6000 X-ray powder diffractometer using Cu K∝ radiation. The instrument is equipped with a fine focus X-ray tube. The tube voltage and amperage were set to 40 kV and 40 mA, respectively. The divergence and scattering slits were set at 10 and the receiving slit was set at 0.15 mm. Diffracted radiation was detected by a NaI scintillation detector. A theta-two theta continuous scan at 3°/min (0.4 sec/0.02° step) from 2.5 to 40° 2θ was used. A silicon standard was analyzed to check the instruments alignment. Data were collected and analyzed using XRD-6000 v. 4.1. Samples were prepared for analysis by placing them in a silicon sample holder or an aluminum holder with silicon insert.

XRPD analyses were performed using an Inel XRG-3000 diffractometer equipped with a Curved Position Sensitive (CPS) detector with a 2θ range of 120°. Real time data were collected using Cu-Kα radiation starting at approximately 4° 2θ at a resolution of 0.03° 2θ. The tube voltage and amperage were set to 40 kV and 30 mA, respectively. The monochromator slit was set at 5 mm by 80 μm. The pattern is displayed from 2.5-40° 2θ. Samples were prepared for analysis by packing them into thin-walled glass capillaries. Each capillary was mounted onto a goniometer head that is motorized to permit spinning of the capillary during data acquisition. The samples were analyzed for 5 or 10 minutes. Instrument calibration were performed using a silicon reference standard.

The following data further describe the polymorphic forms of 2-[4-[[1-[3,5-bis(1,1-dimethylethylhydroxyphenyl]thio]-1-[methylethyl]thio]-2,6-bis(1,1-dimethylethyl)phenoxy] ethanoic acid:

Polymorph A

Form A is a crystalline, unsolvated nonhygroscopic material that melts at approximately 175° C.

X-ray powder diffraction shifts are as follows (see FIG. 1): 5.75, 11.50, 13.02, 17.30, 17.51, 19.78 and 22.90.

Polymorph B

Form B is a crystalline, unsolvated nonhygroscopic acetone hemisolvate that desolvates at approximately 125° C.

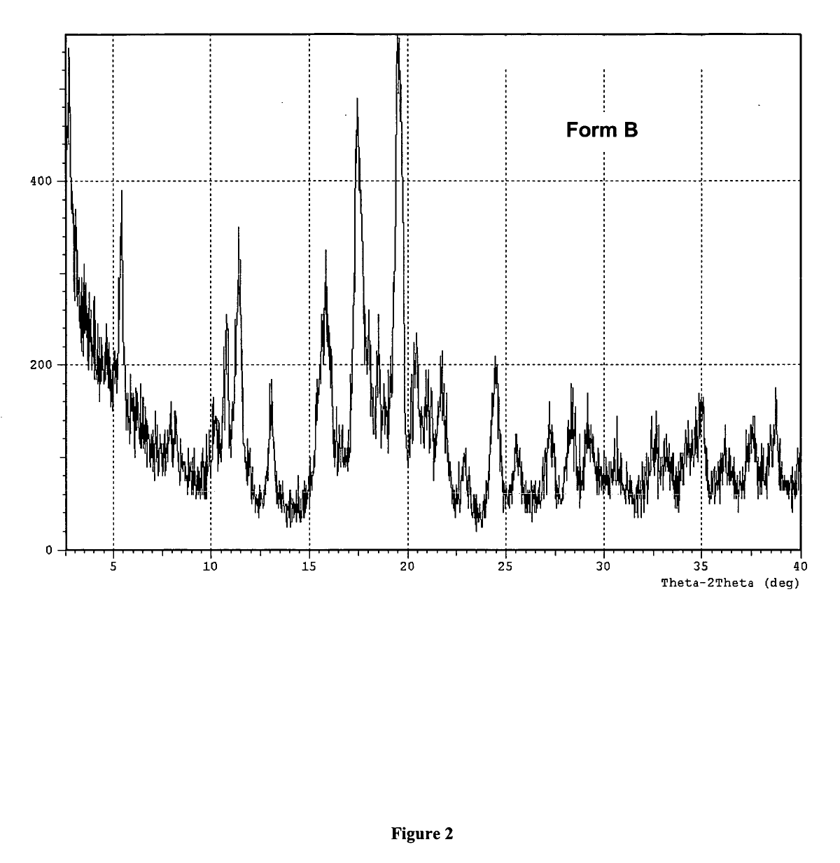

X-ray powder diffraction shifts are as follows (see FIG. 2): 5.50, 10.75, 11.40, 13.00, 15.85, 17.48, 19.50 and 24.48.

Polymorph C

Form C is a crystalline isopropanol hemisolvate that desolvates at approximately 100° C.

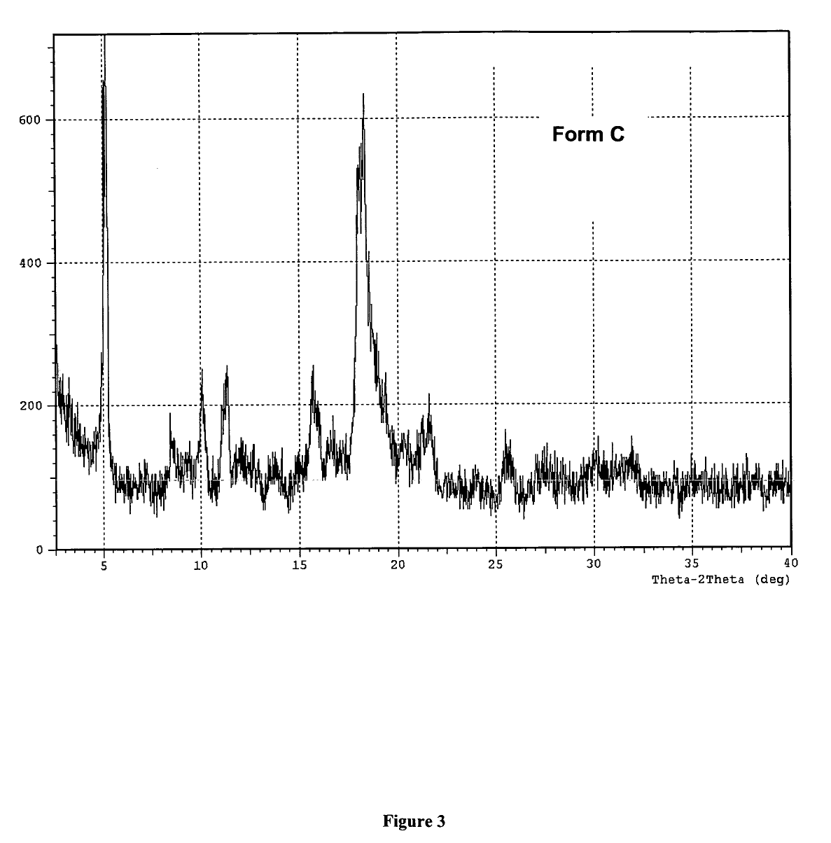

X-ray powder diffraction shifts are as follows (see FIG. 3): 5.10, 10.05, 11.40, 15.75, 18.25 and 25.50.

Polymorph D

Form D is a crystalline dichloromethane solvate that desolvates upon heating.

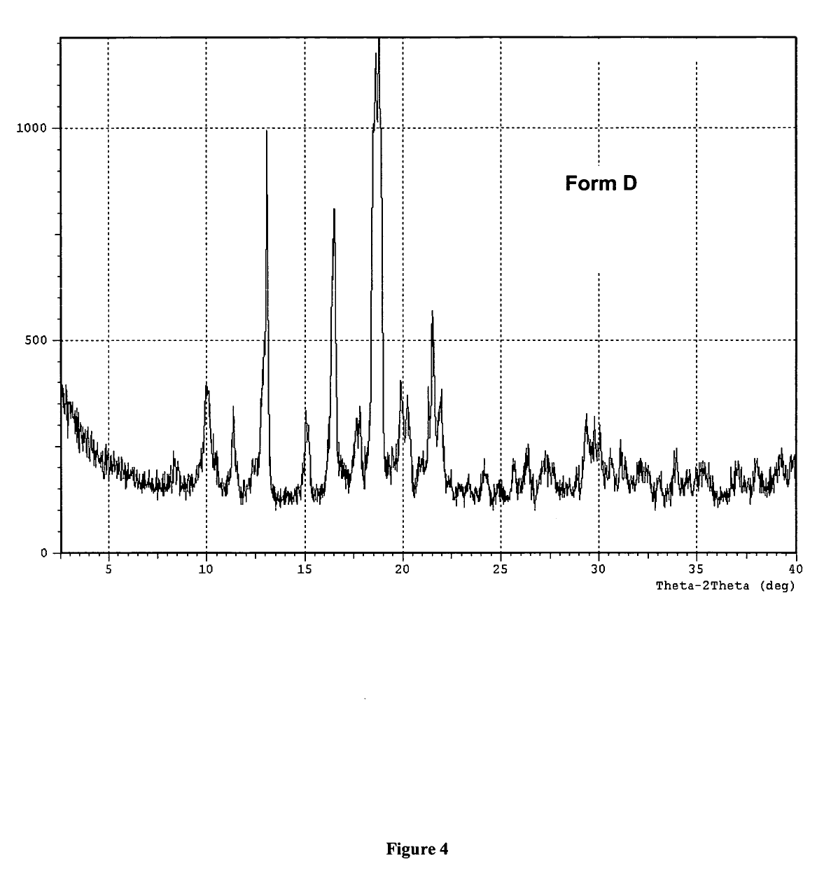

X-ray powder diffraction shifts are as follows (see FIG. 4): 10.00, 11.35, 13.05, 15.10, 16.50, 18.55, 18.75 and 21.50.

Polymorph E

Form E is a crystalline, anhydrous nonhygroscopic material that melts at approximately 163° C.

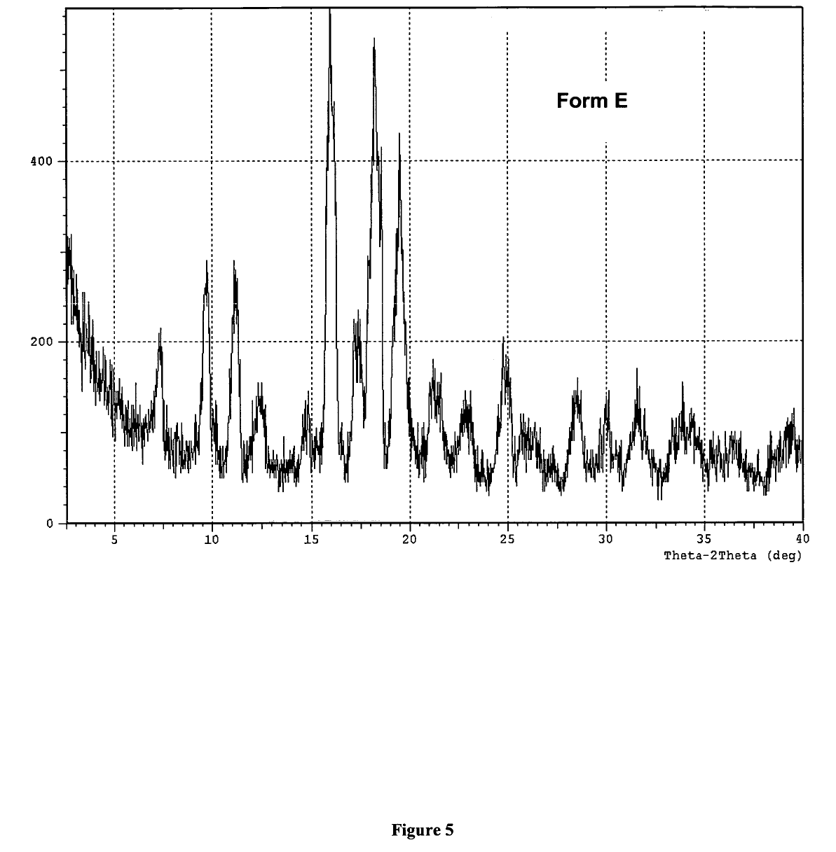

X-ray powder diffraction shifts are as follows (see FIG. 5): 7.40, 9.77, 11.25, 16.00, 18.25, 19.52 and 28.50.

Polymorph F

Form F is a crystalline solvate that desolvates upon heating.

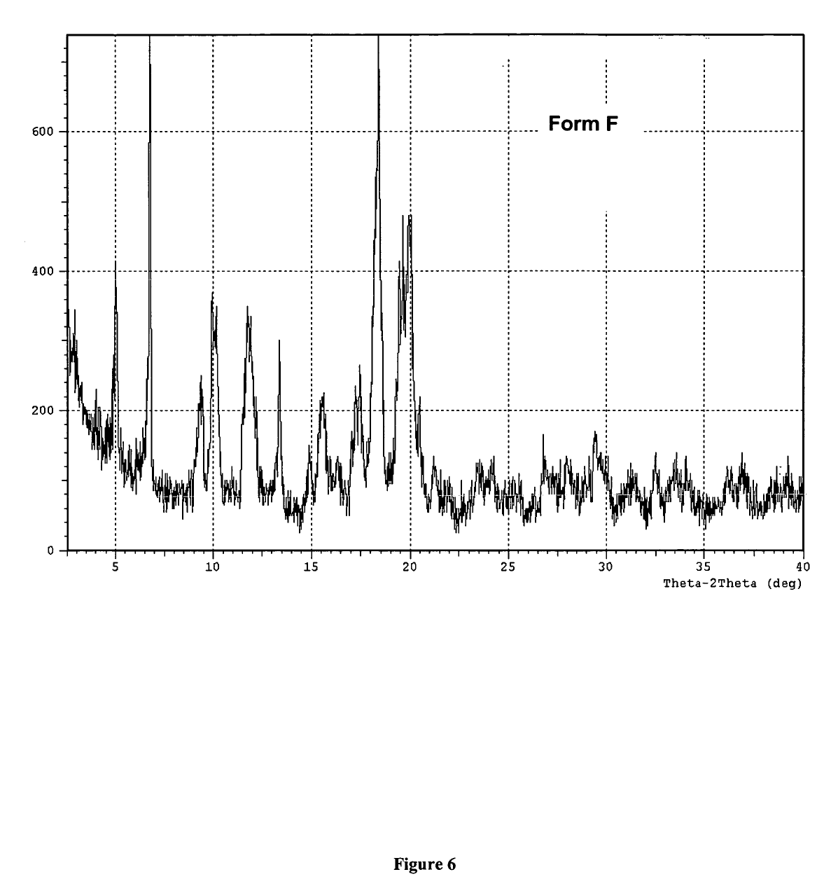

X-ray powder diffraction shifts are as follows (see FIG. 6): 5.00, 6.80, 9.35, 9.95, 10.20, 11.75, 13.45, 14.90, 15.50, 18.40 and 2θ.00.

Polymorph G

Form G is a crystalline, acetonitrile hemisolvate that desolvates at approximately 140° C.

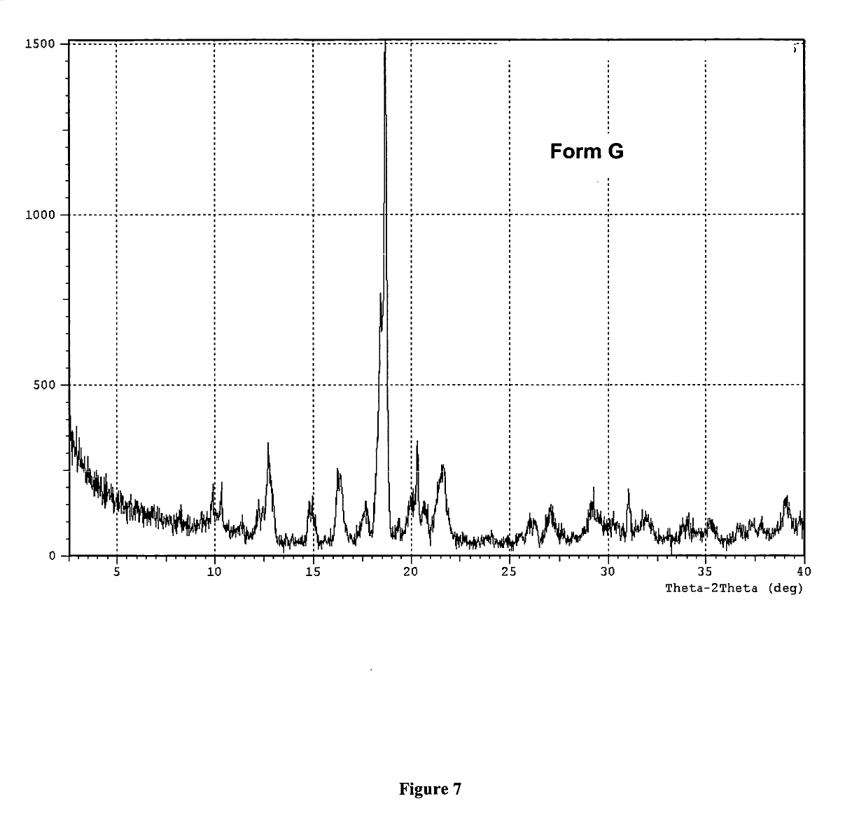

X-ray powder diffraction shifts are as follows (see FIG. 7): 12.80, 14.80, 16.35, 18.55 and 20.35.

Polymorph H

Form H is a solvate that slowly desolvates under ambient conditions.

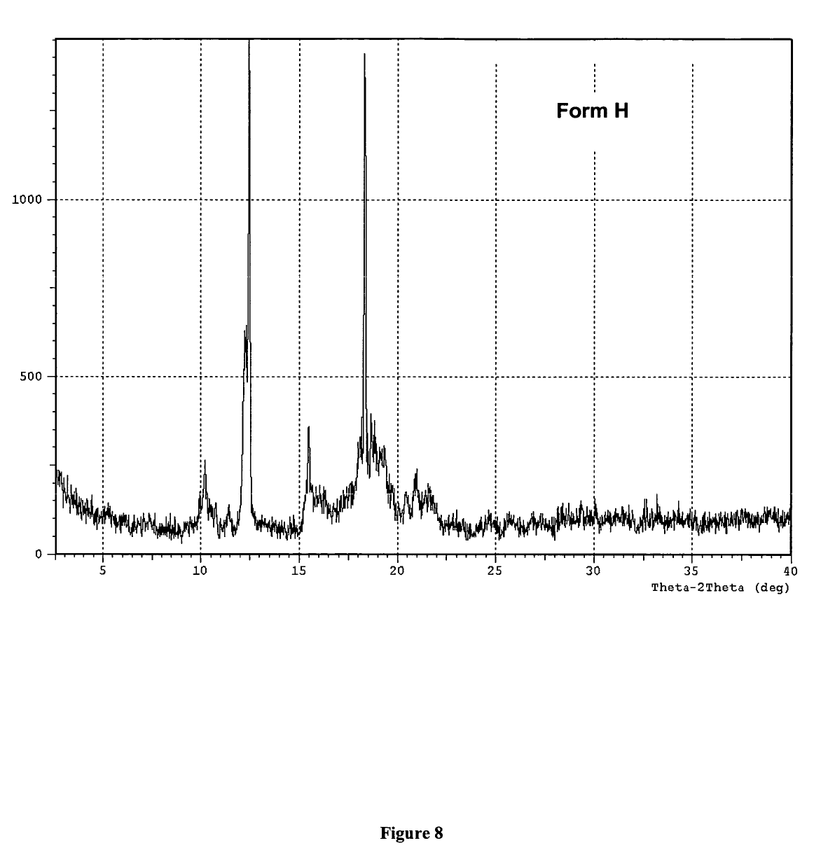

X-ray powder diffraction shifts are as follows (see FIG. 8): 12.25, 12.50, 15.52 and 18.30.

Polymorph I

Form I is an ethyl acetate solvate that desolvates upon heating.

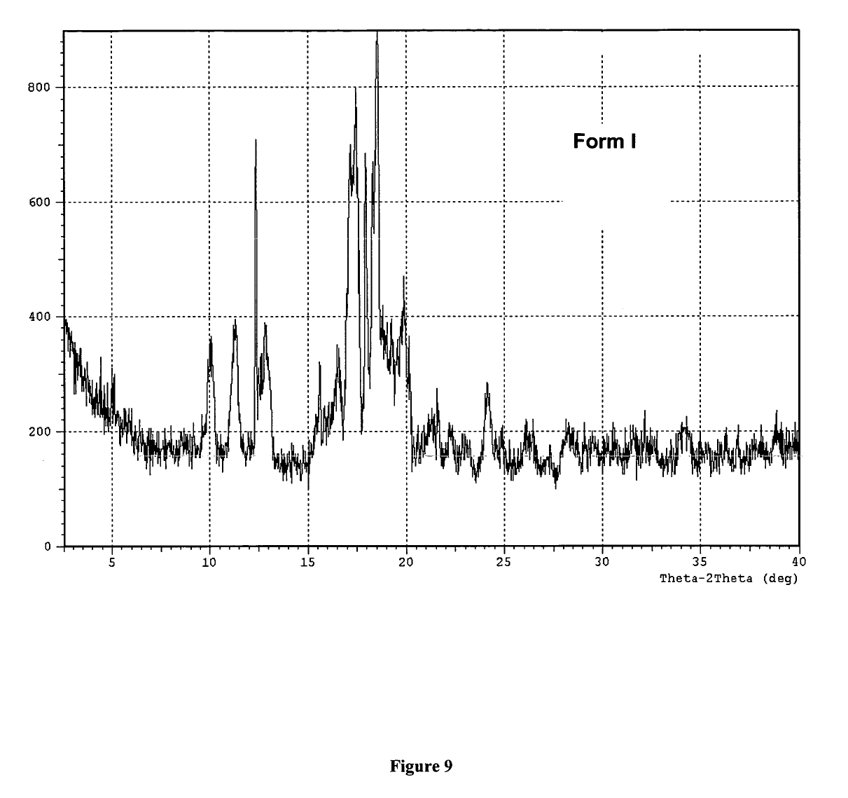

X-ray powder diffraction shifts are as follows (see FIG. 9): 10.10, 11.30, 12.40, 12.95, 17.15, 17.45, 17.95, 18.50 and 19.85.

Polymorph J

Form J—

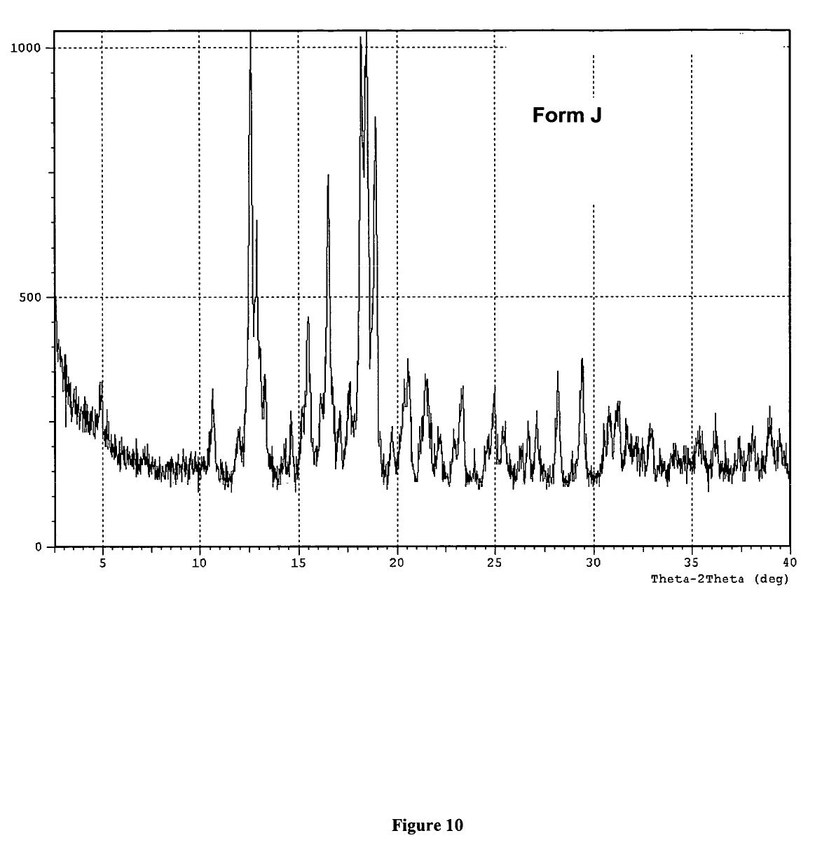

X-ray powder diffraction shifts are as follows (see FIG. 10): 4.98, 10.60, 12.50, 12.90, 15.50, 16.50, 18.15, 18.45, 18.95 and 20.50.

Polymorph K

Form K is a crystalline, unsolvated nonhygroscopic material that melts at approximately 163° C.

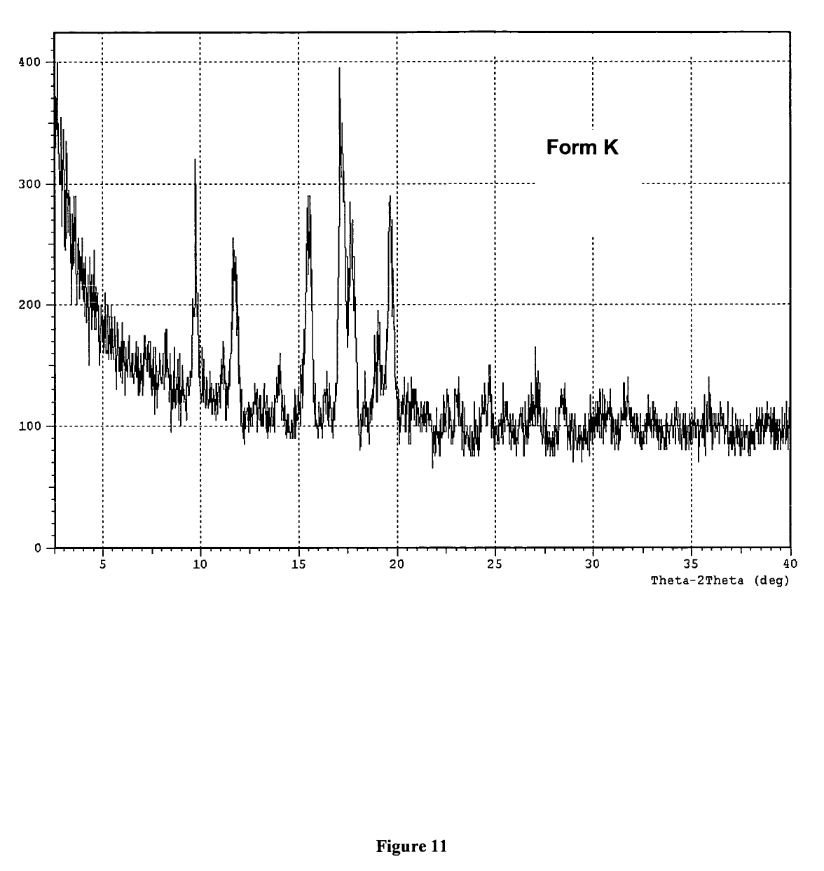

X-ray powder diffraction shifts are as follows (see FIG. 11): 9.65, 11.75, 15.50, 15.58, 17.05, 17.65, 19.55 and 27.10.

Pattern L

Pattern L—

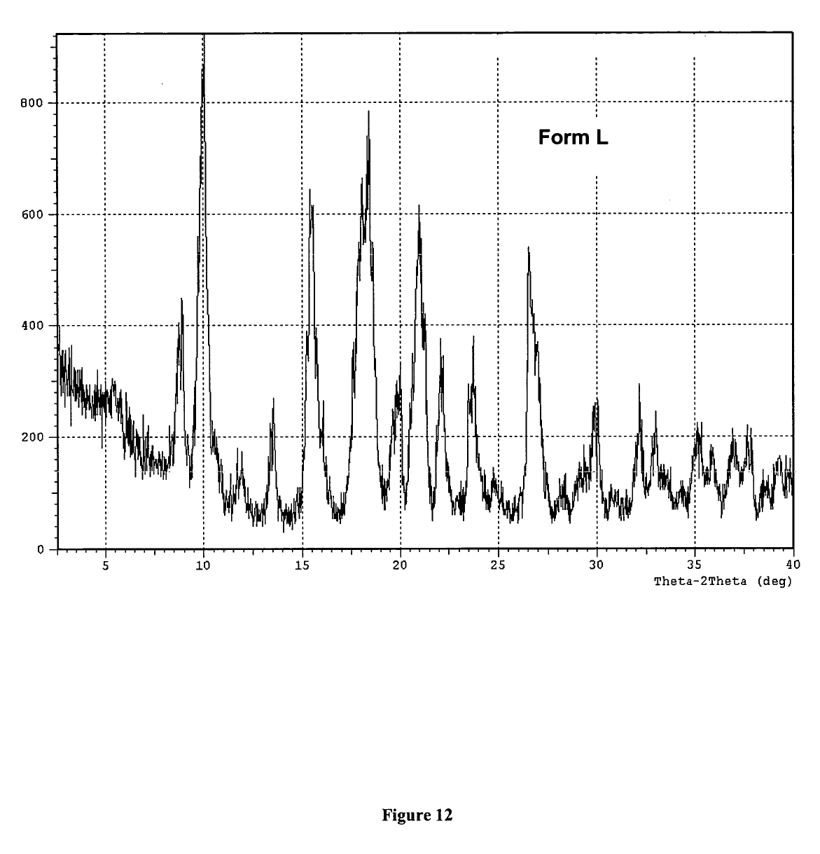

X-ray powder diffraction shifts are as follows (see FIG. 12): 8.80, 10.05, 13.50, 15.48, 18.04, 19.75, 21.00, 22.15, 23.75, 26.53 and 30.00.

Polymorph M

Form M is an acetic acid solvate that desolvates upon heating.

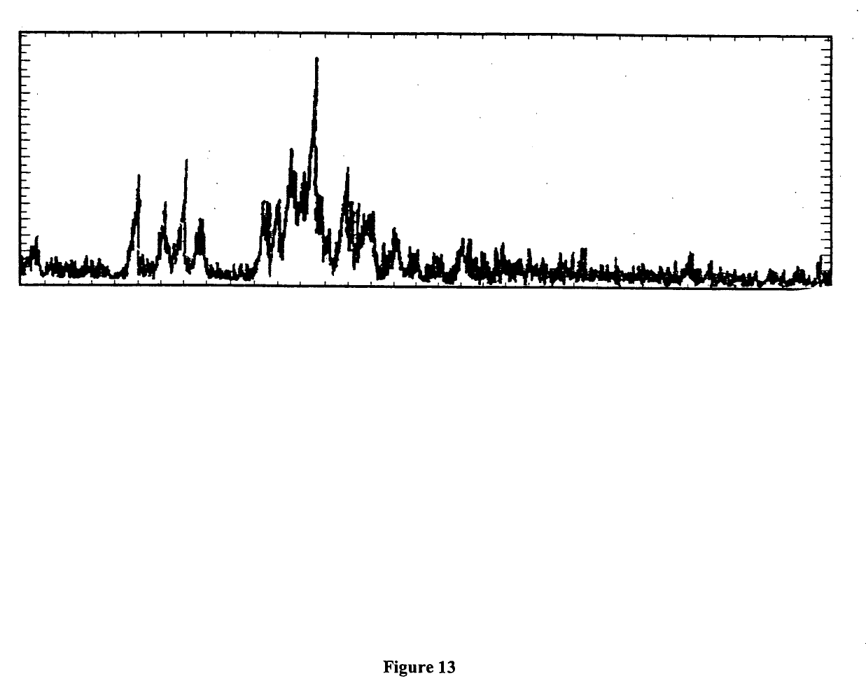

X-ray powder diffraction shifts are as follows (see FIG. 13): 9.90, 11.10, 11.95, 12.75, 15.45, 15.90, 16.45, 17.25 and 18.85.

The following data further describe the polymorphic forms of 2-[4-[[1-[3,5-bis(1,1-dimethylethyl hydroxyphenyl]thio]-1-[methylethyl]thio]-2,6-bis(1,1-dimethylethyl)phenoxy] butanoic acid:

Polymorph Al

Form Al is a crystalline material that melts at approximately 148° C.

X-ray powder diffraction shifts are as follows (see FIG. 14): 5.80, 8.90, 9.33, 10.00, 11.35, 11.75, 13.80, 14.85, 17.70, 19.65, 20.15 and 21.10.

In one particular embodiment of the invention, a process for preparing a water-soluble probucol derivative of the Formula 3 wherein R1 and R2 are the same or different and are independently C1-C6 alkyl, C3-C6 alkenyl or aryl, R3, R4, R5, and R6 are the same or different and are C1-C6 alkyl, Z and Z′ are the same or different and are independently hydrogen, the grou —C1H6-alkyl-C(O)OH or the group —C3H6-alkenyl-C(O)OH wehre Z and Z′ are not both hydrogen is provided including: Step 1: reacting a probucol derivative of the Formula: 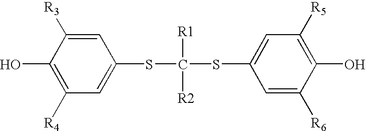
where R1, R2, R3, R4, R5 and R6 are as previously defined with a solution of a compound selected from the group consisting of alkali metal hydroxide, alkali metal alkoxide, alkyl ammonium alkoxide, alkyl ammonium hydroxide and mixtures thereof, thereby forming an ammonium or alkali metal salt mixture of the probucol derivative; Step 2: Reacting the salt mixture with a solution of an alkyl or aryl halo-substituted alkyl or alkenyl or aryl halo-substituted alkenyl carboxylate of the Formula:
Halo-CₙH₂ₙ—C(O)OV or Halo-CₙH_{2n′-2}—C(O)OV
where V is alkyl or aryl; Halo is chloro, bromo or iodo, forming at least one compound of the Formula 2, where R₁, R₂, R₃, R₄, R₅ and R₆ are defined above and Y and Y′ are the same or different and are independently hydrogen or the moiety —CₙH₂ₙ—C(O)OV or the moiety —CₙH_{2n′-2}—C(O)OV where V, is as previously defined, with the proviso that Y and Y′ can not both be hydrogen; Step 3: hydrolyzing the at least one compound of Formula 2 to form a hydrolyzing mixture of the compound of Formula 3; and Step 4: separating a crystalline, polymorphic form of the compound of Formula 3 from the hydrolyzing mixture.

In a particular embodiment of the invention, the compound of Formula 3 is:

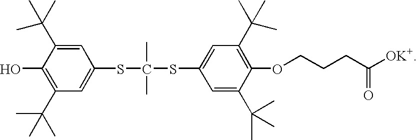

In another particular embodiment of the invention, the compound of Formula 3 is:

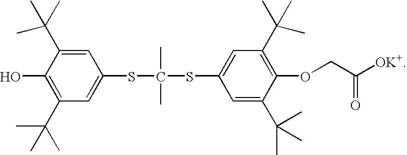

In a more particular embodiment, the compound of Formula 3 is:

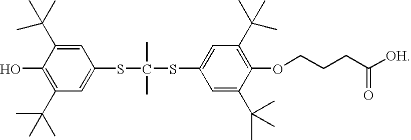

In yet another embodiment, the compound of Formula 3 is:

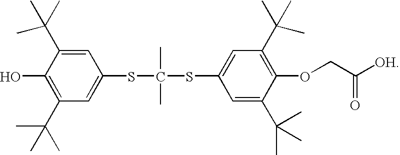

In a further embodiment of the present invention, a pharmaceutical composition for the treatment and/or prophylaxis of inflammatory disorders, abnormal cellular proliferation disorders, atherosclerosis, diabetes, and asthma is described, the composition comprising a polymorphic form of water soluble probucol derivatives as disclosed herein, optionally with a pharmaceutically acceptable carrier or diluent, and optionally with one or more other effective therapeutic agents.

In one embodiment of the invention, a pharmaceutical composition comprising a polymorphic form of 2-[4-[[1-[3,5-bis(1,1-dimethylethyl hydroxyphenyl]thio]-1-[methylethyl]thio]-2,6-bis(1,1-dimethylethyl)phenoxy] ethanoic acid or of 4-[4-[[1-[3,5-bis(1,1-dimethylethyl hydroxyphenyl]thio]-1-[methylethyl]thio]-2,6-bis(1,1-dimethylethyl)phenoxy] butanoic acid, optionally in a pharmaceutically acceptable carrier is provided.

In another embodiment of the invention, a mixture of two or more of the polymorphic forms of 2-[4-[[1-[3,5-bis(1,1-dimethylethyl hydroxyphenyl]thio]-1-[methylethyl]thio]-2,6-bis(1,1-dimethylethyl)phenoxy] ethanoic acid and of 4-[4-[[1-[3,5-bis(1,1-dimethylethyl hydroxyphenyl]thio]-1-[methylethyl]thio]-2,6-bis(1,1-dimethylethyl)phenoxy] butanoic acid, optionally in a pharmaceutically acceptable carrier is provided.

The compositions of the above compositions are in any form that is well known from the prior art, i.e., they may be tablets, capsules, etc. and may contain additionally pharmaceutically components such as sweeteners and the like. The prior art contains numerous examples of such components, methods of administration and the like.

As a further embodiment of the present invention, a method for the treatment of an inflammatory disease in a mammal is described, comprising administering an effective amount of a polymorphic form of a water soluble probucol derivative as described herein, optionally with a pharmaceutically acceptable carrier, excipient or diluent, and optionally in combination and/or alternation with one or more other effective therapeutic agents for the treatment of inflammatory disorders.

Pharmaceutical Formulations may conveniently be presented in unit dosage form and may be prepared by any of the methods well known in the art. In general, the Formulations are prepared by uniformly and intimately bringing into association the active ingredient with liquid carriers or finely divided solid carriers or both and then, if necessary, shaping the product into the desired Formulation.

The compound can be mixed with other active materials that do not impair the desired action, or with materials that supplement the desired action, including other drugs against diabetic vascular disease or ocular inflammatory disease. Solutions or suspensions used for parenteral, intradermal, subcutaneous, or topical application can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS).

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

Additionally, there is provided herein novel polymorphic forms water soluble derivatives of probucol compounds. These polymorphic forms are of the compound 2-[4-[[1-[3,5-bis(1,1-dimethylethyl hydroxyphenyl]thio]-1-[methylethyl]thio]-2,6-bis(1,1-dimethylethyl)phenoxy] ethanoic acid and of 4-[4-[[1-[3,5-bis(1,1-dimethylethyl hydroxyphenyl]thio]-1-[methylethyl]thio]-2,6-bis(1,1-dimethylethyl)phenoxy] ethanoic acid, herein designated as polymorph Form A, polymorph Form B, polymorph Form C, polymorph Form D, polymorph Form E, polymorph Form F, polymorph Form G, polymorph Form H, polymorph Form I, polymorph Form J, polymorph K, polymorph Pattern L and polymorph Form M and of the compound 2-[4-[[1-[3,5-bis(1,1-dimethylethyl hydroxyphenyl]thio]-1-[methylethyl]thio]-2,6-bis(1,1-dimethylethyl)phenoxy] ethanoic acid and of 4-[4-[[1-[3,5-bis(1,1-dimethylethyl hydroxyphenyl]thio]-1-[methylethyl]thio]-2,6-bis(1,1-dimethylethyl)phenoxy] butanoic acid, herein designated as polymorph Form Al which has acceptable stability, and may have enhanced solubility in aqueous fluids and therefore improved bioavailability as compared to the amorphous form of such compounds.

The present invention is described in detail in the examples set forth below which are provided by way of illustration only and therefore should not be considered as limiting the scope of the invention.

### EXAMPLES

### Example 1

### Synthesis of Water Soluble Derivatives of Probucol

In an appropriate vessel, 11 g of potassium tert-butoxide and 50 g probucol are stirred in 225 g N-methylpyrrolidone (NMP) at about 20° C. To this solution is charged 19.6 g ethyl bromoacetate, and the system is stirred for at least one hour. Hydrolysis is accomplished by charging 60 g of 45% potassium hydroxide (KOH) and stirring for at least four hours. The reaction mixture is diluted with 300 mL of water and 60 mL of brine, then extracted at least three times with 150 mL portions of toluene. The washed aqueous NIMIP mixture is charged with 6 mL of 85% aqueous phosphoric acid, then extracted twice with 200 mL portions of toluene. The toluene extracts are washed with 250 g of 2.5% aqueous phosphoric acid, then with 100 g of water. The resultant toluene solution is then successively extracted at least four times with dilute aqueous potassium bicarbonate (1.0-2.5%). Optionally, the toluene solution can be stirred with 60 g of a drying agent and 1 g of activated carbon and then filtered. The resultant toluene solution is washed with 100 g of 2.5% aqueous phosphoric acid, then with 100 g of water. The washed toluene solution is concentrated in vacuo at about 50° C. to a thick oil. The oil is diluted with 220 mL of N-octane and concentrated as before at about 500 C to a volume of about 75 mL. After gradual cooling to about 20° C., the resultant solids are filtered and washed with 50 g of n-octane. Drying in VCJCUO at about 700 C gave 13.8 g of off-white solid 2-[4-[[1-[3,5-bis(1,1-dimethylethyl hydroxyphenyl]thio]-1-[methylethyl]thio]-2,6-bis(1,1-dimethylethyl)phenoxy] ethanoic acid.

### Examples 2-15

Preparation of the Polymorphic Forms of 2-[4-[[1-[3,5-bis(1,1-dimethylethyl hydroxyphenyl]thio]-1-[methylethyl]thio]-2,6-bis(1,1-dimethylethyl)phenoxy] ethanoic acid.

### Example 2

A slurry of about 14 kg of crude (amorphous) 2-[4-[[1-[3,5-bis(1,1-dimethylethylhydroxyphenyl]thio]-1-[methylethyl]thio]-2,6-bis(1,1-dimethylethyl)phenoxy] ethanoic acid obtained by the procedure shown in the above Example 1 and 62 kg of heptane is atmospherically distilled to minimum volume, diluted with 21 kg of heptane and again concentrated to minimum volume by distillation. The resulting slurry is diluted with 26 kg heptane, heated to solution at reflux (90-98° C.) for 1-2 hours, cooled to room temperature (20-25° C.) and stirred for approximately one hour. The resulting slurry is filtered and the solid residue washed with 20 kg heptane. After drying under vacuum at 65° C. for about 12 hours, the white solid polymorphic Form A (XRPD) material has a melting point (capillary) of 169° C.

### Example 3

A further method for preparation of the polymorphic Form A of the above probucol derivative, comprises heating a solution of the amorphous form of said water soluble derivative of the probucol compound in a nonpolar, preferably hydrocarbon, solvent or in a polar solvent such as an ether, chlorinated solvents or in aqueous mixtures for a time and at a temperature sufficient to form said polymorphic Form A and, after cooling, separating said polymorphic Form A from said solution.

Form A may also be prepared by desolvation of polymorphic forms Forms B, D, I and M; and by melting and allowing Form E to recrystallize at room temperature.

### Example 4

The method for preparation of the polymorphic Form B of the above probucol derivative, comprises evaporating a solution of the amorphous compound 2-[4-[[1-[3,5-bis(1,1-dimethylethyl hydroxyphenyl]thio]-1-[methylethyl]thio]-2,6-bis(1,1-dimethylethyl)phenoxy] ethanoic acid from acetone or by conversion of Form A in aqueous acetone.

### Example 5

The method for preparation of the polymorphic Form C of the above probucol derivative, comprises evaporation of the amorphous or any other of the polymorphic forms from a solution in isopropyl alcohol.

### Example 6

The method for preparation of the polymorphic Form D of the above probucol derivative, comprises evaporation of the amorphous or any other of the polymorphic forms from a solution in dichloromethane.

### Example 7

The method for preparation of the polymorphic Form E of the above probucol derivative, comprises preparing a solution of the amorphous or any other of the polymorphic forms from in an acetone solvent and rapidly evaporating the acetone solvent. It may also be prepared by desolvation of Form H.

### Example 8

The method for preparation of the polymorphic Form Form F of the above probucol derivative, comprises preparing a solution of the amorphous or any other of the polymorphic forms in an n-propanol solvent and evaporating the n-propanol solvent.

### Example 9

The method for preparation of the polymorphic Form G of the above probucol derivative, comprises preparing a solution of the amorphous or any other of the polymorphic forms in an acetonitrile solvent and evaporating the acetonitrile solvent.

### Example 10

The method for preparation of the polymorphic Form H of the above probucol derivative, comprises preparing a solution of the amorphous or any other of the polymorphic forms in an acetone solvent and evaporating the acetone solvent.

### Example 11

The method for preparation of the polymorphic Form I of the above probucol derivative, comprises preparing a solution of the amorphous or any other of the polymorphic forms in an ethyl acetate solvent and evaporating the ethyl acetate solvent.

### Example 12

The method for preparation of the polymorphic Form Form J of the above probucol derivative, comprises preparing a solution of the amorphous or any other of the polymorphic forms in a dimethyl sulfoxide solvent and evaporating the dimethyl sulfoxide solvent.

### Example 13

The method for preparation of the polymorphic Form Form K of the above probucol derivative, comprises the desolvation of Forms C, F and g and by stressing of the amorphous compound.

### Example 14

The method for preparation of the polymorphic form Pattern L of the above probucol derivative, comprises preparing a solution of the amorphous or any other of the polymorphic forms in a dimethyl sulfoxide solvent and rapidly evaporating the dimethyl sulfoxide solvent.

### Example 15

The method for preparation of the polymorphic Form M of the above probucol derivative, comprises preparing a solution of the amorphous or any other of the polymorphic forms in an acetic acid solvent and precipitating Form M from such solvent.

### Example 16

### Synthesis of Compound 2

In a 500 mL glass vessel, 5.3 g of potassium tert-butoxide, 25 g probucol are stirred in 93 mL N-methylpyrrolidone (NMP) at about 28° C. to give a clear solution. To this solution is charged 10.5 mL ethyl bromobutyrate. The mixture is heated to about 94° C. for about three hours and cooled again to 0° C.

Hydrolysis is accomplished by charging 22 mL of 45% potassium hydroxide (KOH) and stirring for at least 16 hours. The reaction mixture is diluted with 150 mL of water and 30 mL of aqueous sodium chloride. The aqueous NMP system is washed with 2×75 mL of toluene then treated with 6 mL of 55% aqueous phosphoric acid. The system is then extracted twice with 100 mL portions of toluene. The toluene extracts are washed successively with 125 mL of 2.5% aqueous phosphoric acid, at 0° C. To this mixture is added 22 mL of 45% potassium hydroxide and the system stirred for about 16 hours. To this mixture is added 150 mL of water and 30 mL of saturated sodium chloride. The aqueous NMP system is washed with 2×75 mL of toluene, then treated with 6 mL of 55% phosphoric acid. The system is extracted with 2×100 mL of itoluene, and the toluene extracts are washed successively with 125 mL of aqueous phosphoric acid (2.5%), 50 mL of water and 50 mL portions of dilute aqueous potassium bicarbonate (2.5-10%). Optionally, the toluene solution can be stirred with a drying agent and then filtered. The resultant toluene solution is washed with 100 mL of 2.5% aqueous phosphoric acid, then with 50 mL of water. The washed toluene solution is concentrated in vacuo at about 50° C. to a thick oil. The oil is diluted with 220 mL of n-octane and concentrated as before at about 50° C. to a volume of about 80 mL. After gradual cooling to about 250 C, the resultant solids are filtered and washed with 50 g of n-octane. Drying in vacuo at about 70° C. gave 6.2 g of solid 2-[4-[[1-[3, 5-bis(1,1-dimethylethyl hydroxyphenyl]thio]-1-[methylethyl]thio]-2,6-bis(1,1-dimethylethyl) phenoxy] butanoic acid.

### Examples 17

Preparation of Polymorphic Form Al of 2-[4-[[1-[3,5-bis(1,1-dimethylethyl hydroxyphenyl]thio]-1-[methylethyl]thio]-2,6-bis(1,1-dimethylethyl)phenoxy] butanoic acid.

The method for preparation of the polymorphic Form A of the above probucol derivative, comprises preparing a solution of the amorphous form in MBTE solvent, exchanging the solvent for heptane and precipitating Form Al from such solvent.

1-15. (canceled)

16. A polymorphic form of the compound 2-[4-[[1-13,5-bis(1,1-dimethylethyl hydroxyphenyl]thio]-1-[methylethyl]thio]-2,6-bis(1,1-dimethyl)ethyl)phenoxy] ethanoic acid said compound selected from the group consisting of
(a) Form A, a crystalline, unsolvated nonhygroscopic material that melts at approximately 173° C. and has an X-ray powder diffraction pattern characterized by the shifts: 5.75, 11.50, 13.05, 17.30, 17.51, 19.78, and 22.90;
(b) Form B, a crystalline, unsolvated nonhygroscopic acetone hemisolvate that desolvates at approximately 125° C. and has an X-ray powder diffraction pattern characterized by the shifts: 5.50, 10.75, 11.40, 13.00, 15.85, 17.48, 19.50 and 24.48;
(c) Form M, an acetic acid solvate that desolvates upon heating and has an X-ray powder diffraction pattern characterized by the shifts: 9.90, 11.10, 11.95, 12.75, 15.45, 15.90, 16.45, 17.25 and 18.85.
(d) Form C, a crystalline isopropanol hemisolvate that desolvates at approximately 100° C. and has an X-ray powder diffraction pattern characterized by the following shifts: 5.50, 10.75, 11.40, 13.00, 1585, 17.48, 19.50 and 24.48;
(e) Form D, a crystalline dichloromethane solvate that desolvates upon heating and has an X-ray powder diffraction pattern characterized by the following shifts: 10.00, 11.35, 13.05, 15.10, 16.50, 18.55, 18.75 and 21.50;
(f) Form E, a crystalline, anhydrous nonhygroscopic material that melts at approximately 163° C. and has an X-ray powder diffraction pattern characterized by the following shifts: 7.40, 9.77, 11.25, 16.00, 18.25, 19.52 and 28.50;
(g) Form F, a crystalline solvate that desolvates upon heating and has an X-ray powder diffraction pattern characterized by the following shifts: 5.00, 6.80, 9.35, 9.95, 10.20, 11.75, 13.45, 14.90, 15.50, 18.40 and 20.00;
(h) Form G, a crystalline, acetonitrile hemisolvate that desolvates at approximately 140° C. and has an X-ray powder diffraction pattern characterized by the following shifts: 12.80, 14.80, 16.35, 18.55 and 20.35;
(i) Form H, a solvate that slowly desolvates under ambient conditions and has an X-ray powder diffraction pattern characterized by the following shifts: 12.25, 12.50, 15 52 and 18.30;
(j) Form I, an ethyl acetate solvate that desolvates upon heating and has an X-ray powder diffraction pattern characterized by the following shifts: 10.10, 11.30, 12.40, 12.95, 17.15, 17.45, 17 95, 18.50 and 19.85;
(k) Form J, a dimethyl sulfoxide solvate that desolvates upon heating and has an X-ray powder diffraction pattern characterized by the following shifts: 4.98, 10.60, 12.50, 12.90, 15.50, 16.50, 1815,18.45,18.95 and 20.50;
(l) Form K, being a crystalline, unsolvated nonhygroscopic material that melts at approximately 163° C. and has an X-ray powder diffraction pattern characterized by the following shifts: 9.65, 11.75, 15.50, 15.58, 17.05, 17.65, 19.55 and 27.10; and
(m) Pattern L, having an X-ray powder diffraction pattern characterized by the following shifts: 8.80, 10.05, 13.50, 15.48, 18.04, 19.75, 21.00, 22.15, 23.75, 26.53 and 30.00.

17. The polymorphic form of the compound of claim 16 wherein the form is Form A.

18. A polymorphic form of the compound 2-[4-[[1-[3,5-bis(1,1-dimethylethylhydroxyphenyl] thio]-1-[methylethyl]thio]-2,6-bis(1,1-dimethylethyl)phenoxyl butanoic acid the compound named Form Al and being a crystalline material that melts at approximately 148° C. and has an X-ray powder diffraction pattern characterized by the following shifts: 5.80, 8.90, 9.33, 10.00, 1.35, 11.75, 13.80, 14.85, 17.70, 19.65, 20.15 and 21.10.

19. A pharmaceutical composition comprising a polymorphic form of a compound 2-[4-[[1-13,5-bis(1,1-dimethylethyl hydroxyphenyl]thio]-1-[methylethyl]thio]-2,6-bis(1,1-dimethyl)ethyl)phenoxy] ethanoic acid said compound selected from the group consisting of
(a) Form C, being a crystalline isopropanol hemisolvate that desolvates at approximately 100° C. and has an X-ray powder diffraction pattern characterized by the following shifts: 5.50, 10.75, 11.40, 13.00, 1585, 17.48, 19.50 and 24.48;
(b) Form D, being a crystalline dichloromethane solvate that desolvates upon heating and has an X-ray powder diffraction pattern characterized by the following shifts: 10.00, 11.35, 13.05, 15.10, 16.50, 18.55, 18.75 and 21.50;
(c) Form E, being a crystalline, anhydrous nonhygroscopic material that melts at approximately 163° C. and has an X-ray powder diffraction pattern characterized by the following shifts: 7.40, 9.77, 11.25, 16.00, 18.25, 19.52 and 28.50;
(d) Form F, being a crystalline solvate that desolvates upon heating and has an X-ray powder diffraction pattern characterized by the following shifts: 5.00, 6.80, 9.35, 9.95, 10.20, 11.75, 13.45, 14.90, 15.50, 18.40 and 20.00;
(e) Form G, being a crystalline, acetonitrile hemisolvate that desolvates at approximately 140° C. and has an X-ray powder diffraction pattern characterized by the following shifts: 12.80, 14.80, 16.35, 18.55 and 20.35;
(f) Form H being a solvate that slowly desolvates under ambient conditions and has an X-ray powder diffraction pattern characterized by the following shifts: 12.25, 12.50, 15 52 and 18.30;
(g) Form I being an ethyl acetate solvate that desolvates upon heating and has an X-ray powder diffraction pattern characterized by the following shifts: 10.10, 11.30, 12.40, 12.95, 17.15, 17.45, 17 95, 18.50 and 19.85;
(h) Form J, being a dimethyl sulfoxide solvate that desolvates upon heating and has an X-ray powder diffraction pattern characterized by the following shifts: 4.98, 10.60, 12.50, 12.90, 15.50, 16.50, 1815,18.45,18.95 and 20.50;
(i) Form K, being a crystalline, unsolvated nonhygroscopic material that melts at approximately 163° C. and has an X-ray powder diffraction pattern characterized by the following shifts: 9.65, 11.75, 15.50, 15.58, 17.05, 17.65, 19.55 and 27.10; and
(j) Pattern L, having an X-ray powder diffraction pattern characterized by the following shifts: 8.80, 10.05, 13.50, 15.48, 18.04, 19.75, 21.00, 22.15, 23.75, 26.53 and 30.00.
